(19) United States
(12) Patent Application Publication
(10) Pub. No.: US 2012/0180791 A1
(43) Pub. Date: Jul. 19, 2012

(54) Ventilator Attachment Fitting Usable on an Endotracheal Tube Having an Integrally Formed Suction Lumen and Method of Making And/Or Using the Same

(75) Inventor: Paul Ciccone, Social Circle, GA (US)

(73) Assignee: C. R. Bard, Inc., Murray Hill, NJ (US)

(21) Appl. No.: 13/389,773

(22) PCT Filed: Aug. 18, 2010
(86) PCT No.: PCT/US2010/045915

§ 371 Date: Mar. 29, 2012

Related U.S. Application Data
(60) Provisional application No. 61/235,489, filed on Aug. 20, 2009.

(51) Int. Cl. A61M 16/04; A61M 1/00

(52) U.S. Cl. 128/204.18

(57) ABSTRACT

A ventilation connector (10) for connecting an endotracheal tube (4) to a ventilation source includes a body (11/13). A ventilation port (12) is arranged on the body (11/13) and is configured to connect to the ventilation source. A suction port (15) is arranged on the body (11/13) and is integrally formed with the body (11/13). An interface portion (16/17) is configured to extend into a proximal end (2) of the endotracheal tube (4). A method for intubation using the connector (10) includes inserting at least a portion (3) of endotracheal tube (4) into a trachea, inflating a cuff (8) of the endotracheal tube (4), and supplying gas into a patients lungs via the endotracheal tube (4).

### CROSS REFERENCE TO RELATED APPLICATIONS

The instant application is an International Application based on U.S. provisional application No. 61/235,489, filed Aug. 20, 2009, the disclosure of which is hereby expressly incorporated by reference hereto in its entirety.

### STATEMENT CONCERNING GOVERNMENT INTEREST

Not applicable.

### BACKGROUND OF THE INVENTION

1. Field of Invention

The invention relates generally to the field of medical devices, e.g., a medical tube (e.g., an endotracheal tube) including an inflatable cuff. According to various embodiments, the endotracheal tube utilizes the novel ventilator attachment fitting and/or connector as well as a main tube which utilizes an integrally formed suction lumen and also, in embodiments, an integrally formed inflation lumen.

2. Discussion of Background Information

Conventional methods of endotracheal intubation involve the insertion of a tubular device, e.g., an endotracheal tube, into the trachea. The endotracheal tube typically passes through the trachea and terminates above the carina, allowing gases to be directed through the tube and into the lungs.

A primary objective of this type of treatment is the mechanical ventilation of a patient's lungs, which may be required or appropriate due to the subject's medical condition. In order to create the air pressure necessary to artificially ventilate the lungs, the passageways around the tube are typically sealed, which may be accomplished, e.g., using the inflatable cuff. The cuff is typically located within the trachea about 3-5 centimeters above the carina and is then inflated to expand and seal against the wall of the trachea. This prevents gases from being pumped into the lungs from backing up around the tube.

Although cuffed endotracheal tubes perform an important service, they can allow secretions to collect proximate the cuff and provide a site for the possible accumulation of pathogens. Various methods have been devised for removing such secretions. For example, a small opening may be provided above the cuff with an associated suction lumen. Fluids and/or solids (e.g., secretions) can be periodically or continuously removed through the opening and lumen by suction.

It is also known that cuffed endotracheal tubes often do not self-center within the trachea upon inflation of the cuff. As a result, the suction openings of a particular tube may not be spaced apart from the tracheal wall. For example, due to the curvature of the tube and/or other factors, the suction opening may be located very near the tracheal wall upon cuff inflation. In some instances, the suction opening may actually contact the tracheal wall. In such situations, the tracheal wall membrane may be drawn into the suction opening upon application of a vacuum, thereby occluding the opening. This may prevent the proper removal of secretions from the subglottic space and/or may cause trauma to the tracheal wall.

Known endotracheal tubes also incorporate pigtail tubing to connect the cuff inflation lumen to an inflation syringe and a suction lumen to deliver suctioning into the trachea from a suction source. These pigtails typically separate from the main tube at a point below where the main tube is typically cut to fit a particular patient. While the pigtail for cuff inflation is small, the pigtail for suctioning is larger and can cause space issues within the limited space of the patient oral cavity. This is especially the case during, e.g., oral care and inspections.

It would be beneficial to have a main tube include an integrally formed suction lumen and that this suction lumen is configured so that when the main tube is cut, an end of the suction lumen is substantially flush with an end of the inflation lumen. It would also be beneficial to have a connector that can be connected more easily to a main tube of this type, especially after the tube is cut to size or otherwise cut to a length that is tailored to a particular patient.

### SUMMARY OF THE INVENTION

Accordingly to one non-limiting embodiment of the invention, there is provided a ventilation connector for connecting an endotracheal tube to a ventilation source which overcomes one or more of the deficiencies noted above.

Accordingly to one non-limiting embodiment of the invention, there is provided a ventilation connector for connecting an endotracheal tube to a ventilation source, wherein the connector comprises a body, a ventilation port arranged on the body and being configured to connect to the ventilation source, a suction port arranged on the body and being integrally formed with the body, and an interface portion configured to extend into a proximal end of the endotracheal tube.

The body may be a one-piece plastic body. The connector may further comprise a flange arranged between the suction port and the interface portion. The flange may comprise slots or openings. The flange may be integrally formed with the body. The connector may further comprise a flange arranged between the ventilation port and the interface portion. The flange may comprise oppositely arranged slots or openings. The interface portion may be integrally formed with the body.

The interface portion may comprise at least one of first and second projecting portions, first and second spaced-apart projecting portions, first and second projecting portions projection by different amounts, first and second projecting portions having different axial lengths, first and second projecting portions having different cross-sectional shapes, first and second projecting portions having different cross-sectional sizes, first and second projecting portions having different sized internal passages, and first and second projecting portions having different shaped internal passages.

The first projecting portion may be configured to extend into a ventilation lumen of the endotracheal tube and the second projecting portion may be configured to extend into a suction lumen of the endotracheal tube. The endotracheal tube may comprise an integrally formed ventilation lumen and an integrally formed suction lumen and the interface portion comprises first and second spaced-apart projecting portions. The first projecting portion may be configured to extend into the ventilation lumen of the endotracheal tube and the second projecting portion may be configured to extend into the suction lumen of the endotracheal tube.

The integrally formed ventilation lumen and the integrally formed suction lumen may each have a central axis which is generally parallel to a center axis of the endotracheal tube. The integrally formed ventilation lumen and the integrally formed suction lumen may each have a central axis which is offset from a center axis of the endotracheal tube.

The endotracheal tube may comprise an integrally formed ventilation lumen, an integrally formed suction lumen, and an integrally formed inflation lumen, and the interface portion comprises first and second spaced-apart projecting portions. The first projecting portion may be configured to extend into the ventilation lumen of the endotracheal tube and the second projecting portion may be configured to extend into the suction lumen of the endotracheal tube. The integrally formed ventilation lumen, the integrally formed suction lumen, and the integrally formed inflation lumen may each have a central axis which is generally parallel to a center axis of the endotracheal tube. The integrally formed ventilation lumen, the integrally formed suction lumen, and the integrally formed inflation lumen may each have a central axis which is offset from a center axis of the endotracheal tube.

The endotracheal tube may comprise an integrally formed ventilation lumen, a smaller integrally formed suction lumen, and an even smaller integrally formed inflation lumen, and the interface portion comprises first and second spaced-apart projecting portions. The first projecting portion may be configured to extend into the ventilation lumen of the endotracheal tube and the second projecting portion may be configured to extend into the suction lumen of the endotracheal tube. The integrally formed ventilation lumen, the integrally formed suction lumen, and the integrally formed inflation lumen may each have a central axis which is generally parallel to a center axis of the endotracheal tube. The integrally formed ventilation lumen, the integrally formed suction lumen, and the integrally formed inflation lumen may each have a central axis which is offset from a center axis of the endotracheal tube.

The endotracheal tube may comprise an integrally formed non-circular ventilation lumen, a smaller integrally formed circular suction lumen, and an even smaller non-circular integrally formed inflation lumen, and the interface portion may comprise first and second spaced-apart projecting portions. The endotracheal tube may comprise an integrally formed non-circular ventilation lumen, a smaller integrally formed generally circular suction lumen, and an even smaller oval integrally formed inflation lumen, and the interface portion may comprise first and second spaced-apart projecting portions.

The endotracheal tube may have a generally oval cross-section. The endotracheal tube may comprise an integrally formed non-circular ventilation lumen defining a first lumen cross-sectional area, an integrally formed generally circular suction lumen defining a second lumen cross-sectional area, and a generally oval integrally formed inflation lumen defining a third lumen cross-sectional area.

The endotracheal tube may comprise an integrally formed non-circular ventilation lumen defining a first lumen cross-sectional area, an integrally formed generally circular suction lumen defining a second lumen cross-sectional area, and a generally oval integrally formed inflation lumen defining a third lumen cross-sectional area. The second lumen cross-sectional area may be less than the first lumen cross-sectional area by a factor of between 2 and 5 times, and the third lumen cross-sectional area is smaller than the second lumen cross-sectional area by a factor of between 3 and 10 times. The second lumen cross-sectional area may be less than the first lumen cross-sectional area by a factor of more than 3 times, and the third lumen cross-sectional area is smaller than the second lumen cross-sectional area by a factor of more than 5 times.

The endotracheal tube may comprise an inflatable cuff. The endotracheal tube may comprise a suction opening arranged adjacent the inflatable cuff and between the cuff and the body. The endotracheal tube may comprise a tapered distal end.

The invention also provides for a method for intubation using the connector described above, wherein the method comprises inserting at least a portion of endotracheal tube into a trachea, inflating a cuff of the endotracheal tube, and supplying gas into a patient's lungs via the endotracheal tube.

The method may further comprise suctioning matter through an opening in the endotracheal tube located adjacent the cuff.

The invention also provides for a method of making the connector described above, wherein the method comprises forming the connector as a one-piece member.

The invention also provides for an endotracheal tube assembly comprising a connector of the type described above and which comprises first and second projecting portions and an endotracheal tube comprising an integrally formed ventilation lumen and at least one of an integrally formed suction lumen and an integrally formed inflation lumen. The first projecting portion is configured to extend into the ventilation lumen of the endotracheal tube and the second projecting portion is configured to extend into the suction lumen of the endotracheal tube.

The integrally formed ventilation lumen, the integrally formed suction lumen, and the integrally formed inflation lumen may each have a central axis which is generally parallel to a center axis of the endotracheal tube. The integrally formed ventilation lumen, the integrally formed suction lumen, and the integrally formed inflation lumen may each have a central axis which is offset from a center axis of the endotracheal tube.

The invention also provides for an endotracheal tube assembly comprising a connector of the type described above and which comprises first and second projecting portions and an endotracheal tube comprising an integrally formed ventilation lumen, an integrally formed suction lumen, and an integrally formed inflation lumen. The first projecting portion is configured to extend into the ventilation lumen of the endotracheal tube and the second projecting portion is configured to extend into the suction lumen of the endotracheal tube.

The integrally formed ventilation lumen, the integrally formed suction lumen, and the integrally formed inflation lumen may each have a central axis which is generally parallel to a center axis of the endotracheal tube. The integrally formed ventilation lumen, the integrally formed suction lumen, and the integrally formed inflation lumen may each have a central axis which is offset from a center axis of the endotracheal tube. The integrally formed ventilation lumen may be non-circular and define a first lumen cross-sectional area, the integrally formed suction lumen may be generally circular and define a second lumen cross-sectional area, and the integrally formed inflation lumen may be generally oval and define a third lumen cross-sectional area. The second lumen cross-sectional area may be less than the first lumen cross-sectional area by a factor of between 2 and 5 times, and the third lumen cross-sectional area may be smaller than the second lumen cross-sectional area by a factor of between 3 and 10 times. The second lumen cross-sectional area may be less than the first lumen cross-sectional area by a factor of more than 3 times, and the third lumen cross-sectional area may be smaller than the second lumen cross-sectional area by a factor of more than 5 times.

The invention also provides for a method of making an endotracheal tube, wherein the method comprises cutting a main tube to a desired length and installing a connector on the main tube. The connector and/or main tube may utilize at least one feature described above.

### BRIEF DESCRIPTION OF DRAWINGS OF THE EXEMPLARY EMBODIMENTS

FIG. 1 shows a side perspective view of a ventilator attachment fitting and/or connector in accordance with an exemplary embodiment of the invention;

FIG. 2 shows a side cross-section view of the connector of FIG. 1;

FIG. 3 shows a top sectional view through lines in FIG. 2;

FIG. 4 shows a side perspective view of a main tube which can be used with the connector of FIG. 1. The main tube has an integrally formed ventilation lumen, an integrally formed suction lumen, and an integrally formed inflation lumen;

FIG. 5 shows a side perspective view of an endotracheal tube assembly in accordance with an exemplary embodiment of the invention. The assembly utilizes the connector of FIG. 1, the tube of FIG. 4, and the inflation cuff of FIG. 10;

FIG. 6 shows a rear side view of the assembly shown in FIG. 5;

FIG. 7 shows a left side view of the assembly shown in FIG. 5;

FIG. 8 shows a side cross-sectional view through lines VIII-VIII in FIG. 6;

FIG. 9 shows a cross-sectional view through lines XI-XI in FIG. 7;

FIG. 10 shows an enlarged side view of the inflatable cuff used in the assembly of FIG. 5 in accordance with one exemplary embodiment of the invention. The cuff is shown in an inflated position and in a bent configuration; and

FIG. 11 shows a side cross-sectional view similar to that shown in FIG. 7 and illustrating one exemplary way in which an inflation tube and inflation device can be coupled to the integrally formed inflation lumen.

### DETAILED DESCRIPTION OF THE INVENTION

The following description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will enable one skilled in the art to make and use the invention, and describes several embodiments, adaptations, variations, alternatives and uses of the invention, including what is presently believed to be the best mode of carrying out the invention.

As used herein, the reference terms “proximal” and “distal” (proximal being closer than distal) refer to proximity with respect to a health care professional inserting the endotracheal tube into a patient. For example, the region or section of the endotracheal tube that is closest to the health care professional during insertion is referred to herein as “proximal,” while a region or section of the endotracheal tube closest to the patient's lungs is referred to as “distal.”

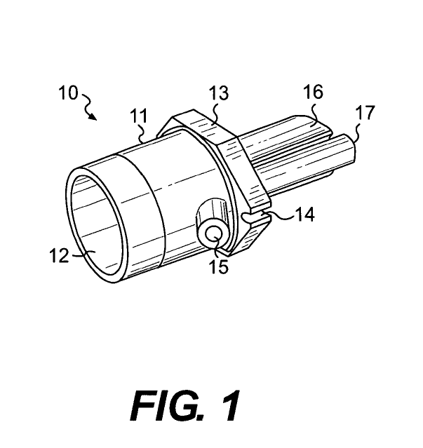

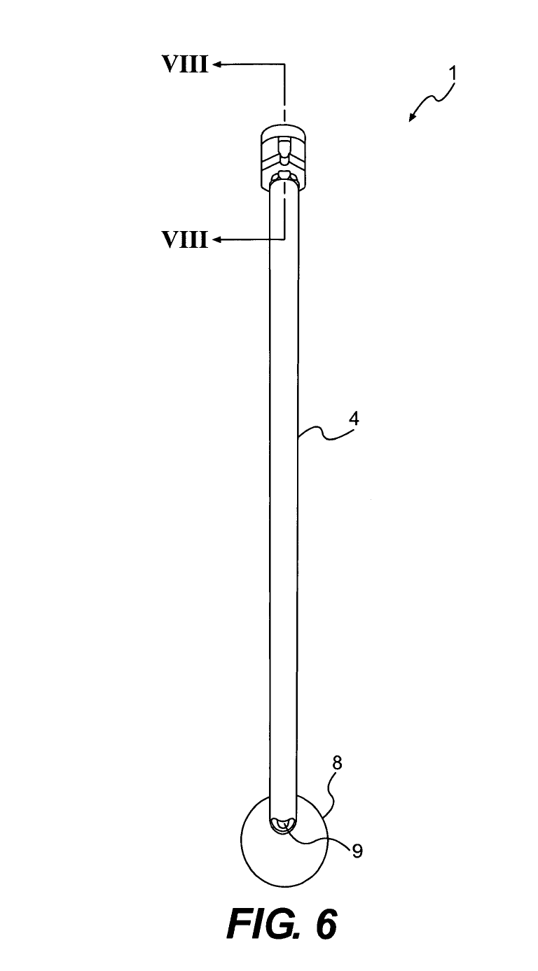

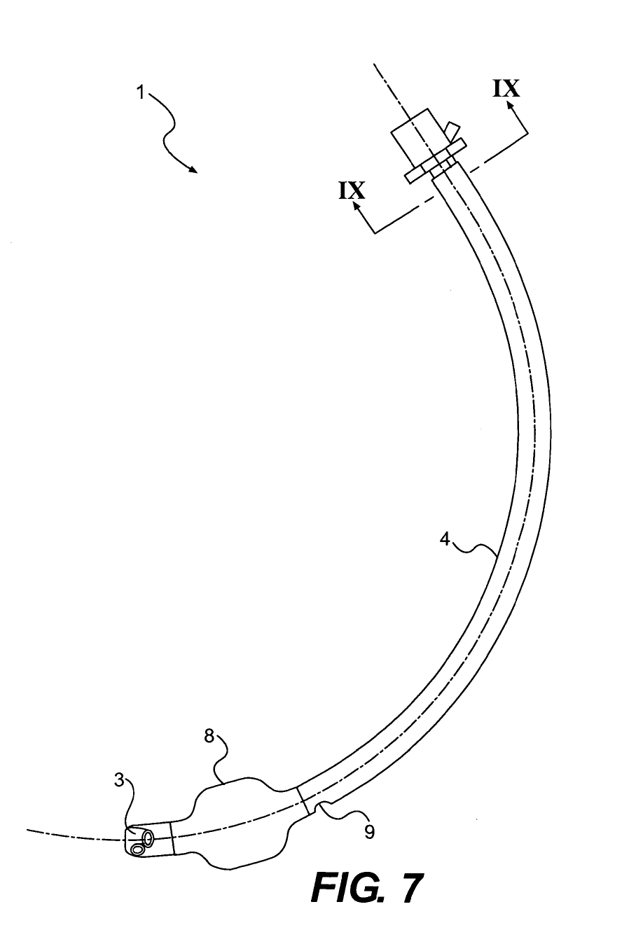

FIGS. 1-11 show non-limiting or exemplary embodiments of the invention wherein like numbers refer to same and like parts. The present invention broadly relates to medical tubes (e.g., endotracheal, tracheostomy, or oropharyngeal tubes or other tubes or catheters) adapted to be intubated into one or more passageways (e.g., the trachea and/or pharynx) of a patient, subject or user in connection with a medical procedure. For example, certain embodiments are directed toward endotracheal tubes inserted into a subject's trachea to facilitate mechanical ventilation of the lungs. Certain embodiments include tubes having an improved configuration for periodic removal of fluids and/or solids that collect adjacent an inflatable cuff used to seal, secure, and/or position the tube against the tracheal wall. The endotracheal tube of the invention, in embodiments, includes a connector of the type described herein which advantageously connects the main tube to a ventilation source. As used throughout this document, the terms “subject”, “patient” or “user” may include any human or other animal.

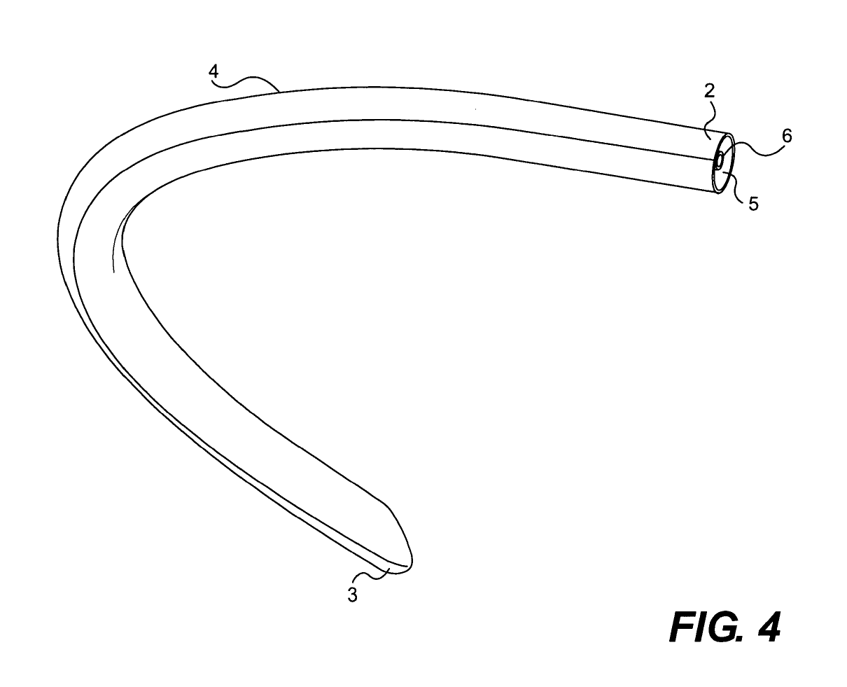

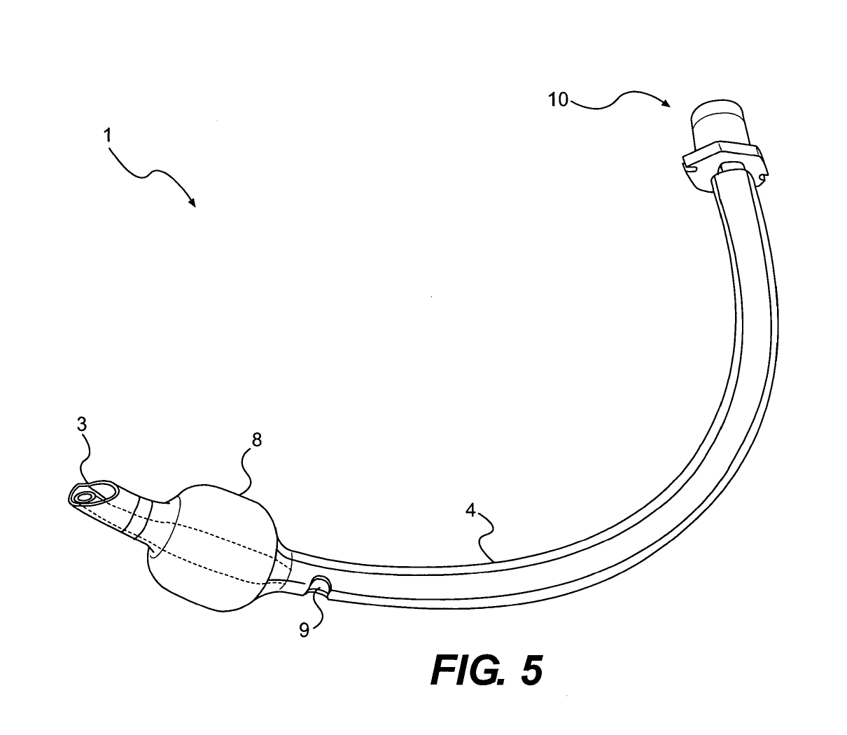

With reference to FIG. 1, there is shown one non-limiting embodiment of an attachment fitting or connector 10 in accordance with the invention. The connector 10 is, in embodiments, used with an endotracheal tube assembly of the type shown in FIG. 5. As is apparent from FIG. 1, the connector 10 is a one-piece member. By way of non-limiting example, the connector 10 can be made of any medical grade plastic such as, e.g., polypropylene. The connector 10 includes a generally cylindrical section 11 which is configured to interface with a tube of a ventilation source (not shown). In this regard, the section 11 includes a generally cylindrical main connecting port 12 which can receive therein a portion of the tubing of the ventilation source. The member 10 also includes a flange 13 which can be used in ensuring a good connection between the member 10 and the tubing of the ventilation source. In this regard, the flange 13 includes slots or openings 14 which can be arranged opposite one another and which receive therein connecting members, e.g., fasteners. (not shown). The member 10 also includes an insertable interface section which is configured to extend into one end of a main tube 4 (see FIG. 4). In this regard, the insertable interface section includes a ventilation interface portion 16 which is sized and shaped to extend into an integrally formed ventilation lumen 5 (see FIG. 4) and an integrally formed suction interface portion 17 which is sized and shaped to extend into an integrally formed suction lumen 6 (see FIG. 4). The member 10 additionally also includes an integrally formed suction port 15 which is in fluid communication with the portion 17 and which is configured to be connected to tubing of a source of suction (not shown). In embodiments, the suction port 15 is oriented at an angle relative to a center axis of the section 11.

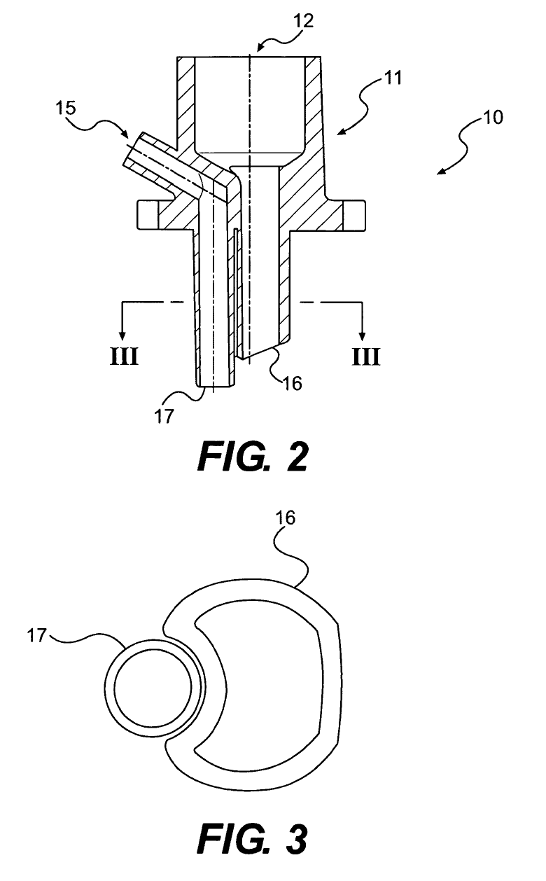

With reference to FIG. 2, it can be seen that the suction port 15 and suction interface portion 17 form a generally uniform lumen or passage for suction. In this regard, the cross-section shape of this lumen can be generally circular (see FIG. 3). The ventilation port 12 and the ventilation interface portion 16 form a lumen or passage for ventilation gases. In this regard, the cross-section shape of this lumen need not be circular and, in embodiments, has a non-circular shape such as the shape shown in, e.g., FIG. 3. The interface sections 16 and 17 are also, in embodiments, separated from one another and has a different axial length (e.g., section 17 being longer than section 16) so that each portion can sealingly fit into the respective lumens 5 and 6, and can more easily be inserted therein. As the each of the interface sections 16 and 17 have a respective central axis which is offset from a main central axis of the main tube 4 (see FIGS. 4 and 9), the integrally formed ventilation lumen 5 and the integrally formed suction lumen 6 each have a central axis which is generally parallel to a center axis of the main tube 4.

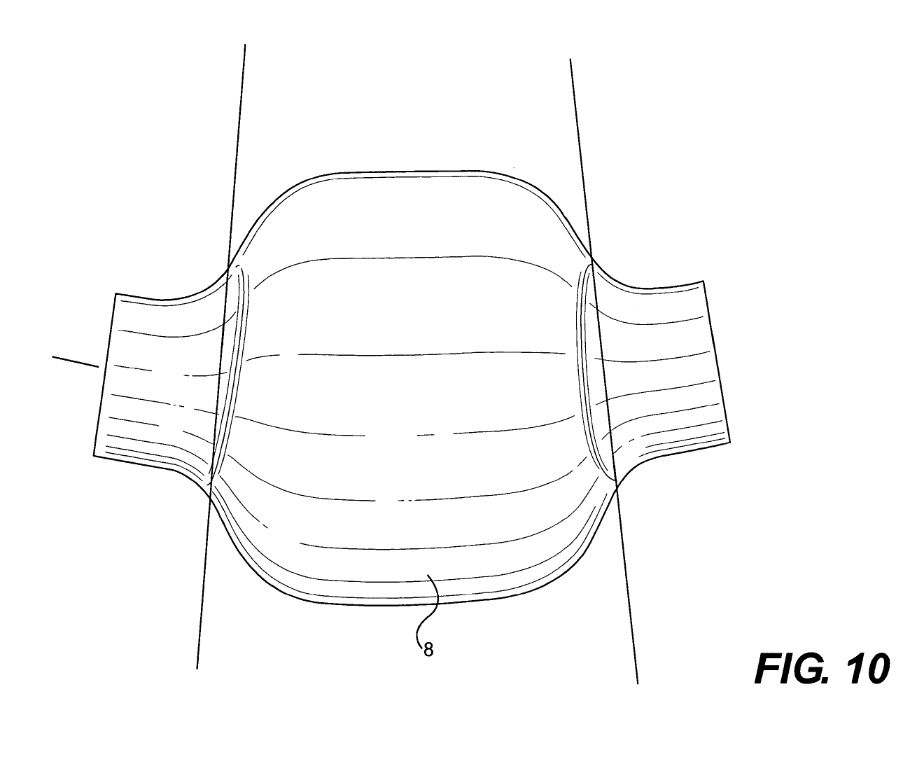

With reference to FIG. 4-7, there is shown one non-limiting embodiment of a main tube 4 which can be used with the connector 10. The main tube 4 is, in embodiments, a one-piece member. By way of non-limiting example, the main tube 4 can be made of any medical grade plastic such as, e.g., polyvinylchloride. The main tube 4 can also be made substantially transparent or translucent. The main tube 4 includes a connector end 2 and a tapered end 3. The connector end 2 is sized, configured, and shaped to receive therein the interface sections 16 and 17. The main tube 4 also includes an integrally formed ventilation lumen 5 and the integrally formed suction lumen 6. The lumens 5 and 6 can have the cross-sectional shapes shown in FIG. 9. Each lumen 5 and 6 extends from end 2 to end 3, and, in embodiments, has a central axis which is offset from a center axis of the main tube 4. In embodiments, the tapered end 3 is oriented so as to be generally perpendicular to the cross-sectional plane shown in FIG. 2. In this regard, a suction opening 9 opens into the suction lumen 6 and is also oriented generally perpendicular to the tapered end 3. The opening 9 allows secretions collected proximate the cuff 8 to be removed or suctioned out. The main tube 4 also includes an inflation cuff 8. With reference to FIG. 10 and by way of non-limiting example, the cuff 8 can have any shape and can be made of any medical grade plastic such as, e.g., polyvinylchloride.

In FIG. 4, it can be seen that when the end 2 of the main tube 4 is cut to a desired length, the lumens 5 and 6 are flush with the end 2. Installing the connector 10 on the main tube 4 can therefore be accomplished more easily by inserting portion 17 into lumen 6 and portion 16 into lumen 5.

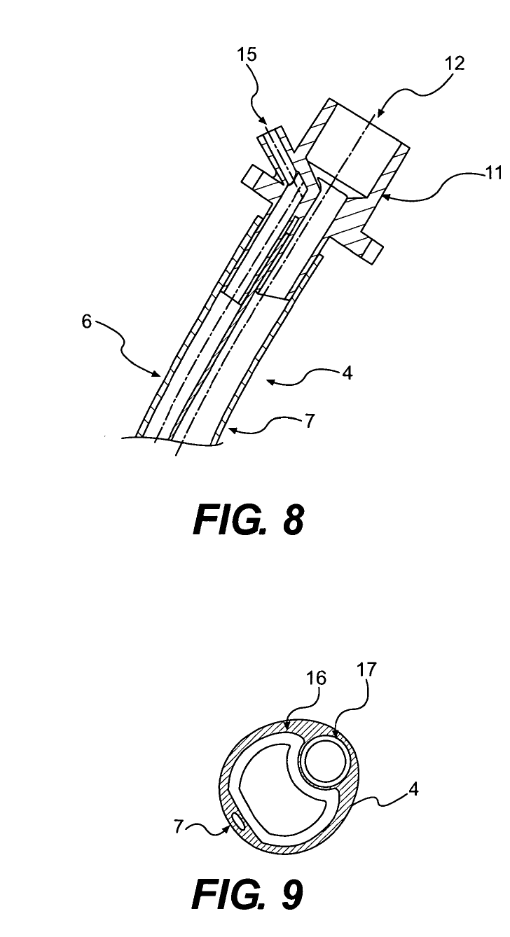

With reference to FIGS. 8 and 9, it can be seen that the main tube 4 can have a generally oval cross-section shape and can also include an integrally formed inflation lumen 7. By way of non-limiting example, the lumen 7 can have a generally oval cross-section shape. The lumen 7 is in fluid communication with an inflation opening (not shown) and allows a gas to pass into and through the lumen 7 so as to inflate the cuff 8. The inflation lumen 7 has a center axis which is offset from a main axis of the main tube 4.

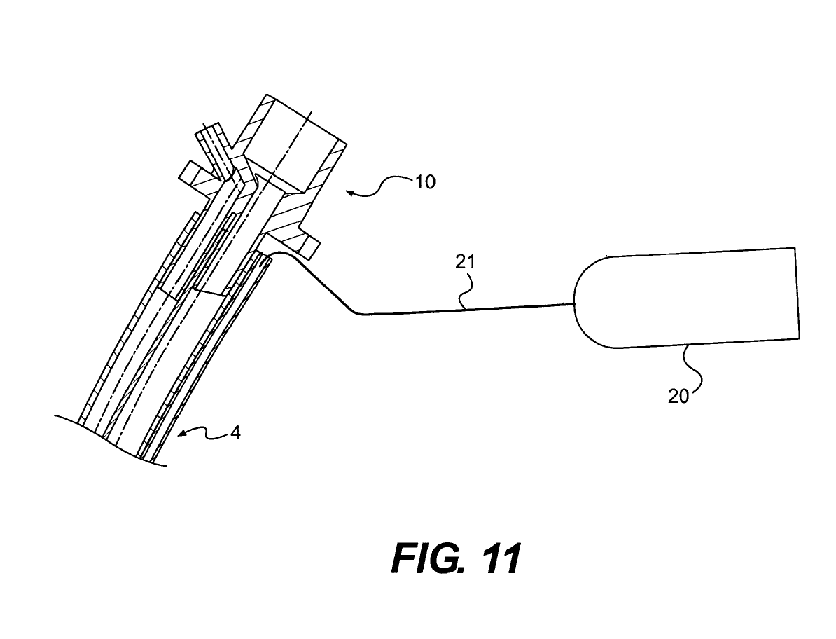

With reference to FIG. 11, it can be seen that an inflation device 20 can, in embodiments, be connected to the inflation lumen 7 so as to allow the cuff 8 to be inflated. A tube 21 is used to connect the inflation lumen 7 to the inflation device 20. Any conventionally known types of such tubes 21 and devices 20 can be utilized.

The members 10 and 4 may, in embodiments, be constructed from a suitable polymeric material, such as polyethylene or polypropylene. The components of the assembly disclosed herein can also be made from various well-known materials. The components 10 and 4 can be molded or extruded according to well-known manufacturing techniques.

Materials commonly used to make the member 4 include, but are not limited to thermoplastic elastomers (TPE). In embodiments, materials which are environmentally green in nature and have no extractables can be utilized. Other materials include, but are not limited to natural rubber latexes (available, for example, from Guthrie, Inc., Tucson, Ariz.; Firestone, Inc., Akron, Ohio; and Centrotrade USA, Virginia Beach, Va.), silicones (available, for example, from GE Silicones, Waterford, N.Y., Wacker Silicones, Adrian, Mich.; and Dow Corning, Inc., Midland, Mich.), polyvinyl chlorides (available, for example, from Kaneka Corp., Inc., New York, N.Y.), polyurethanes (available, for example, from Bayer, Inc., Toronto, Ontario, Rohm & Haas Company, Philadelphia, Pa.; and Ortec, Inc., Greenville, S.C.), plastisols (available, for example, from G S Industries, Bassett, Va.), polyvinyl acetate, (available, for example from Acetex Corp., Vancouver, British Columbia) and methacrylate copolymers (available, for example, from Heveatex, Inc., Fall River, Mass.). Natural rubber latexes, polyurethanes, and silicones are preferred materials. Any combination of the foregoing materials may also be used in making catheters. In one embodiment, a rubberize layer that includes latex and a methacrylate is used with build up and finish layers that include latex but not methacrylate. In another embodiment, a polyurethane rubberize layer is used with latex build up and finish layers. In another embodiment, a polyvinyl acetate and latex rubberize layer is used with latex build up and finish layers. Each of the foregoing embodiments in which specific Young's Modulus values are specified may be used with any material.

The invention also provides for a method for intubation using the assembly of FIGS. 5-7, and specifically using the connector 10, which includes inserting at least a portion 3 of an endotracheal tube 4 into a trachea, inflating a cuff 8 of the endotracheal tube 4, and supplying gas into a patient's lungs via the endotracheal tube 4. The method can further include suctioning matter through an opening 9 in the endotracheal tube 4 located adjacent the cuff 8.

The invention also provides for a method of making a device for intubation shown FIGS. 5-7, and specifically making the connector 10, which includes forming the connector 10 as a one-piece member and having the general configuration shown in FIG. 1.

The invention also provides for a method of making a device for intubation shown FIGS. 5-7 which includes cutting a main tube 4 to a desired length and installing a connector 10 on the main tube 4. The connector 10 and/or main tube 4 may utilize at least one feature described above.

This invention has been described and specific examples of the invention have been portrayed. While the invention has been described in terms of particular variations and illustrative figures, those of ordinary skill in the art will recognize that the invention is not limited to the variations of figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well. Finally, all publications and patent applications cited in this specification are herein incorporated by reference in their entirety as if each individual publication or patent application were specifically and individually put forth herein.

1. A ventilation connector for connecting an endotracheal tube to a ventilation source, the connector comprising:
a body;
a ventilation port arranged on the body and being configured to connect to the ventilation source;
a suction port arranged on the body and being integrally formed with the body; and
an interface portion configured to extend into a proximal end of the endotracheal tube.

2. The connector of claim 1, wherein the body is a one-piece plastic body.

3. The connector of claim 1, further comprising a flange arranged between the suction port and the interface portion.

5. The connector of claim 4, wherein the flange comprises slots or openings.

6. The connector of claim 4, wherein the flange is integrally formed with the body.

7. The connector of claim 1, further comprising a flange arranged between the ventilation port and the interface portion.

8. The connector of claim 7, wherein the flange comprises oppositely arranged slots or openings.

9. The connector of claim 1, wherein the interface portion is integrally formed with the body.

10. (canceled)

11. The connector of claim 1, wherein the interface portion includes a first projecting portion to extend into a ventilation lumen of the endotracheal tube and a second projecting portion to extend into a suction lumen of the endotracheal tube.

12-36. (canceled)

37. An endotracheal tube assembly comprising:
a connector comprising:
a body;
a ventilation port arranged on the body to connect to a ventilation source;
a suction port integrally formed with the body; and
a first projecting portion in fluid communication with the ventilation port and a second projecting portion in fluid communication with the suction port; and
an endotracheal tube comprising an integrally formed ventilation lumen, an integrally formed suction lumen, and an integrally formed inflation lumen, wherein the first projecting portion extends into the ventilation lumen of the endotracheal tube and the second projecting portion extends into the suction lumen of the endotracheal tube.

38. The assembly of claim 37, wherein the integrally formed ventilation lumen, the integrally formed suction lumen, and the integrally formed inflation lumen each have a central axis which is generally parallel to a center axis of the endotracheal tube.

39. The assembly of claim 37, wherein the integrally formed ventilation lumen, the integrally formed suction lumen, and the integrally formed inflation lumen each have a central axis which is offset from a center axis of the endotracheal tube.

40-42. (canceled)

43. The assembly of claim 37, wherein the integrally formed ventilation lumen is non-circular and defines a first lumen cross-sectional area, the integrally formed suction lumen is generally circular and defines a second lumen cross-sectional area, and the integrally formed inflation lumen is generally oval and defines a third lumen cross-sectional area.

44. The assembly of claim 43, wherein the second lumen cross-sectional area is less than the first lumen cross-sectional area by a factor of between 2 and 5 times, and the third lumen cross-sectional area is smaller than the second lumen cross-sectional area by a factor of between 3 and 10 times.

45. The assembly of claim 43, wherein the second lumen cross-sectional area is less than the first lumen cross-sectional area by a factor of more than 3 times, and the third lumen cross-sectional area is smaller than the second lumen cross-sectional area by a factor of more than 5 times.

46-47. (canceled)

48. The assembly of claim 37, wherein the connector further comprises a flange arranged between the suction port and the first and second projecting portions.

49. The assembly of claim 48, wherein the flange comprises oppositely arranged slots or openings.

50. A one-piece ventilation connector for an endotracheal tube, comprising:
a ventilation port;
a suction port separated from the ventilation port;
a first projecting portion in fluid communication with the ventilation port and having a first axial length;
a second projecting portion in fluid communication with the suction port and having a second axial length different from the first axial length, the second projecting portion spaced apart from the first projecting portion; and
a flange positioned between the ventilation port and the first and second projecting portions.

51. The assembly of claim 50, wherein the first projecting portion has a first cross-sectional shape and the second projecting portion has a second cross-sectional shape different from the first cross-sectional shape.
